(19) United States
(12) Patent Application Publication
(10) Pub. No.: US 2005/0245471 A1
(43) Pub. Date: Nov. 3, 2005

(54) Combination products for use in antitumoral treatment

(76) Inventors: Jean-Marc Balloul, Lingolsheim (FR); Suzy Scholl, Paris (FR); Jerome Lacoste, Grenoble (FR)

Correspondence Address: BUCHANAN INGERSOLL PC;(INCLUDING BURNS, DOANE, SWECKER & MATHIS), POST OFFICE BOX 1404, ALEXANDRIA, VA, 22313-1404, US

(21) Appl. No.: 10/500,709

(22) PCT Filed: Jan. 3, 2003
(86) PCT No.: PCT/FR03/00007

§ 371 Date: May 17, 2005

(30) Foreign Application Priority Data
Jan. 3, 2002 (FR) 02/00029

(51) Int. Cl. A61K 48/00

(52) U.S. Cl. 514/44

(57) ABSTRACT

The invention concerns combination products comprising (i) at least a substance capable of inhibiting CSF-1 activity and/or at least a nucleic acid, comprising at least a sequence coding for a substance capable of inhibiting CSF-1 activity and (ii) at least a substance having a cytotoxic activity and/or at least a nucleic acid, comprising at least a sequence coding for a substance having cytotoxic activity. The invention also concerns oligonucleotides capable of inhibiting CSF-1 expression. The invention is particular useful for implementing an antitumoral treatment.

This invention relates to combination products comprising (i) at least one substance capable of inhibiting the activity of CSF-1 and/or at least one nucleic acid, comprising at least one sequence coding for a substance capable of inhibiting the activity of CSF-1 and (ii) at least one substance with a cytotoxic activity and/or at least one nucleic acid, comprising at least one sequence coding for a substance with a cytotoxic activity. This invention also relates to oligonucleotides capable of inhibiting the expression of CSF-1. This invention is particularly useful for the application of an anti-tumour treatment.

At the moment, the most encouraging results obtained in the context of anti-tumour treatments relate to combined treatments that associate a treatment based on chemical compounds (chemotherapy) and a treatment based on the use of radiation (radiotherapy). Apart from the severe side effects that patients experience with this type of treatment, 10 to 50% of relapses are observed (depending on how early the treatment is applied).

Moreover, work carried out in the field of cancer has resulted in suggestions to adapt genic therapy protocols to 5 anti-tumour therapy. For example, in this respect it is worth mentioning the work done by Meneguzzi et al. (1991, Virology, 181, 61-69) related to immunisation against tumour cells using a recombining vector of the vaccine expressing the E6 and E7 genes of the type 16 human papilloma virus (see also patents U.S. Pat. No. 5,744,133 or U.S. Pat. No. 6,007,806) or Leroy et al. (1998, Res. Immunol. 149: 681-684) that demonstrated that the production of cytokines on tumour sites after intra-tumour administration of recombining viral vectors enabled the induction of an immune response associated with inhibition of tumour growth. We can also mention results obtained in genetically modified cells capable of expressing IL2 (Roschlitz et al., 199, Cancer Gene Ther., 6, 271-281) or viral vectors expressing a tumour antigen and an, interleukin (Bizouarne et al., 1996, In “Breast Cancer Advances in Biology and Therapeutics”, F. Calvo, M. Crepin and H. Magdalenat eds., 303-308—for a review, see Zhang, 1999, Cancer Gene Therapy, 6, 113-138).

However, the anti-tumour response observed in the context of these various treatments is encouraging, but it is not better than clinical responses observed following conventional treatments (i.e. chemotherapy, radiotherapy).

Consequently, it is desirable to have new products and/or new methods capable firstly of implementing efficient anti-tumour treatments, that are easy to implement, that enable prolonged control of the tumour volume and an increase in the survival rate of treated patients, with few or no side effects other than the required cytotoxic activity.

The CSF-1 (Colony Stimulating Factor 1) is a cytokine expressed particularly by monocytes, it is a differentiation, growth and survival factor of these cells. Its receptor is a product of proto-oncogene c-fms (Sherr et al., 1990, Blood, 75, 1-12). CSF-1 is also expressed physiologically in the human endometrium and placenta and by some tumour cells. Work done by immunohistochemistry and in situ hybridisation has demonstrated specificity of the expression of CSF-1 in invasive tumour cells while such production is not observed in intra-canal or non-invasive tumour cells (Scholl et all. J. Natl. Cancer. Inst., 1994, 86, 120-126: J. Cellular, Biochem., 1992, 50, 350-356; Mol. Carcinog., 1993, 7207-211). Production of CSF-1 by invasive tumour cells corresponds particularly to an increase in its concentration in the plasma of patients, that can then be more than 1000 pg/ml compared with less than 300 pg/ml in normal subjects. This type of concentration demonstrates an advanced stage of the disease and an unfavourable short term prognostic (Scholl et al., Br. J. Cancer, 1994, 69, 342-346; Scholl et al., Breaste Cancer Res. Treat. 1996 39:275-283).

Moreover, it has been demonstrated that CSF-1 stimulates mobility and invasiveness of tumour cells (Dorsch et al, Eur. J. Imm., 1993; 23, 186-90; Wang et al J. Immunol, 1988, 141, 575-579; Filderman et al., Cancer Research, 1992, 52, 3661-3666). The CSF-1 also has a chemotactical effect on precursors in the myeloid line, which facilitates infiltration of monocytes in the tumour. However, the presence of these monocytes is not sufficient to observe destruction of the tumour by the immune system. (Dorsch et al., 1993, Eur, J. Immunol, 23, 186-190). It appears that at the high serum contents commonly found at patients suffering from tumours of the breast, ovaries or pancreas, CSF-1 orients the differentiation of these monocytes into microphages and not into dendritic cells capable of presenting tumoural antigens and thus initiating an efficient cytotoxic immune response directed against tumour cells (Scholl et al., 1996, Breast Cancer. Res. Treat., 39, 275-283; Baron et al, J. Cell. Sci., 2000, 114, 999-1010).

This application relates to combination products to induce the death of tumour cells and control the concentration of CSF-1 and/or the activity of CFS-1 in treated patients. The association of these two properties is particularly interesting in the context of an anti-tumour treatment. The applicants have demonstrated that patients with the best clinical responses to an anti-tumour treatment consisting of immunising them against tumour cells using a vector coding for MUC-1 mucin, were those for which the serum content of CSF-1 was lowest. More particularly, this type of combination product provides a means of inhibiting or delaying cell proliferation by inducing specific death of cells, particularly tumour cells, a better presentation of antigens and/or a better stimulation of immune cells of the host organism. This invention offers an advantageous and efficient alternative to techniques according to prior art, particularly to treat cancer in man or animals.

Thus, this invention relates to a combination product including:
(i) at least one substance capable of inhibiting the activity of CSF-1 and/or at least one nucleic acid including at least one sequence coding for a substance capable of inhibiting the activity of CSF-1 and;
(ii) at least one substance with at least one cytotoxic activity and/or at least one nucleic acid, comprising at least one sequence coding for a substance with at least one cytotoxic activity.

“Substance capable of inhibiting the activity of CSF-1” means substances capable of inhibiting the expression of CSF-1 and substances capable of inhibiting the bond of CFS-1 to its receptor.

“Capable of inhibiting the expression of CSF-1” means that the action of the said substance reduces the quantity of CSF-1 produced by the target cell. In particular, the capacity of a substance to inhibit the expression of CSF-1 may be checked in vitro by bringing the cells expressing CSF-1 into contact with a substance capable of inhibiting the expression of CSF-1, then by measuring the quantity of CSF-1 expressed by the said cell (Birchenall-Roberts et al., J. Immunol. 1990, 145, 3290-3296).

According to one preferred embodiment of the invention, the substance capable of inhibiting the CSF-1 activity is an oligonucleotide.

One of the oligonucleotides capable of inhibiting the expression of CSF-1 that is particularly worth mentioning is the oligonucleotide described by Birchenall-Roberts et al (J. immunol., 1990, 145, 3290-3296). This oligonucleotide is capable of hybridising the initiation codon of the CFS-1 sequence coding (i.e. the region between the nucleotide in position 97 and the nucleotide in position 99 included in SEQ ID No. 1), efficiently inhibits the expression of CFS-1 by the target cells.

The applicants have also identified new oligonucleotides capable of inhibiting the expression of CSF-1.

Thus, this invention also relates to an oligonucleotide capable of inhibiting the expression of CSF-1, from 8 to 100 nucleotides long, characterised in that it is capable of hybridising the region between the nucleotide in position 121 and the nucleotide in position 450 inclusive, preferably between the nucleotide in position 131 and the nucleotide in position 391 inclusive, and even better between the nucleotide in position 135 and the nucleotide in position 152 inclusive, or between the nucleotide in position 284 and the nucleotide in position 301 inclusive, or between the nucleotide in position 341 and the nucleotide in position 358 inclusive of SEQ ID No. 1.

The term oligonucleotide refers to a single strand nucleotides polymer. Nucleotides are well known to those skilled in the art, they are composed of at least one nucleic base, at least one -ose group and at least one phosphate group.

Oligonucleotides according to the invention may be modified, particularly to improve their stability in vivo, their bond to the target sequence and/or their capture by cells.

In particular, the modified oligonucleotides that can be used in the context of this invention include oligonucleotides comprising one or several phosphorothioate, phosphotriester and/or methylphosphonate bonds. According to one preferred embodiment, the oligonucleotide according to the invention will include one or several phosphorothioate bonds.

The modified oligonucleotides also include oligonucleotides comprising one or several derivatives of adenine, guanine, cytosine, thymine and/or uracil. These derivatives include but are not limited to hypoxanthine, 5-(1-propynyl)-2′-deoxycytidine, 5-(1-propynyl)-2′deoxyuridine, 6-methyladenine, 5-methylcytosine, 5-bromouracil, 5-Hydroxymethyluracil, 8-azaguanine, 7-deazaguanine and 2,6-diaminopurine. According to one preferred embodiment of the invention, the oligonucleotide according to the invention includes one or several 5-(1-propynyl)-2′deoxyuridines and/or one or several 5-(propynyl)-2′-deoxycytidines.

The modified oligonucleotides also include oligonucleotides for which one or several -ose groups have been substituted. “Substituted -ose group” means -ose groups for which the hydrogen or hydroxyl in position 2′, 3′ and/or 5′ is replaced by another chemical function. According to one preferred embodiment of the invention, the oligonucleotide according to the invention comprises one or several 2′-methoxyethoxyl, 2′-propoxy and/or 2′-fluoro radicals.

Other modified oligonucleotides are known to those skilled in the art and can be used in the context of this invention. In particular, note the oligonucleotides described in patent U.S. Pat. No. 6,238,921, the text of which is included here by reference.

Oligonucleotides may be prepared using techniques well known to those skilled in the art, particularly using solid phase synthesis techniques (see Sproat et al. 1984, Solid-phase synthesis of oligodeoxyribonucleotides by the phosphotriester method, in Oligonucleotide Synthesis—A practical approach, Gait, M. J. Ed., IRL Press, Oxford p. 83-115 for a review).

“Capable of hybridising” means that the oligonucleotide according to the invention is capable of bonding specifically to a target sequence. Hybridisation is the consequence of hydrogen bonds between firstly nucleotides of the oligonucleotide, and secondly nucleotides in the target sequence. Before the bond between two nucleotides is possible, the two nucleotides must be complementary. Those skilled in the art know that it is not necessary for 100% of nucleotides composing an oligonucleotide to be complementary to nucleotides in the target sequence, for hybridisation to occur. According to one embodiment of the invention, more than 80% of the nucleotides in the oligonucleotide according to the invention will be complementary to nucleotides in the target sequence, and preferably this percentage will be more than 90%, and ideally 100%.

Hybridisation between an oligonucleotide and a target sequence may be verified using methods well known to those skilled in the art. In particular, methods described by Southern et al (Ciba Found. Symp., 1997, 209, 38-44) are worth mentioning.

Those skilled in the art will be capable of determining the sequence of an oligonucleotide capable of hybridising a given target sequence, and they could use software designed for this purpose (Song et al., Acta Pharmacol Sin., 2000, 21, 80-86).

“Target sequence” means a genomic DNA sequence coding for a polypeptide for which it is required to modulate the expression, the genomic DNA sequence(s) involved in regulation of the transcription of the said sequence coding for a polypeptide for which it is required to modulate the expression, and the transcribed mRNAs for these genomic DNA sequences.

Advantageously, the oligonucleotide according to the invention comprises 8 to 30 nucleotides, preferably 12 to 25 nucleotides, and ideally between 16 and 19 nucleotides.

In the special case in which the oligonucleotide according to the invention is composed solely of deoxyribonucleic acids, it could be transcribed from a nucleic acid sequence. Thus, the invention also relates to a nucleic acid comprising a sequence coding for an oligonucleotide according to the invention.

Substances capable of inhibiting the CSF-1 bond to its receptor include for example anti-bodies, peptides, lipids or sugars capable of bonding to CFS-1 or its receptor.

According to one preferred embodiment, the substance capable of inhibiting the bond of. CSF-1 to its receptor is an antibody. An “antibody” means polyclonal antibodies, monoclonal antibodies, chimeric antibodies, scFv and fragments of antibodies. More particularly, antibody “fragments” mean fragments F(ab)2, Fab′, Fab, Fv, sFv (Blazar et al., 1997, J. of Immunology 159, 5821-5833; Bird et al., 1988, Science 242, 423-426) and Babs (Ward et al., 1989, Nature 341, 544) of a native antibody, regardless of whether it is of polyclonal or monoclonal origin (for example see Monoclonal Antibodies: A manual of Techniques and Applications H. Zola (CRC Press, 1988) and Monoclonal Hybridoma Antibodies: Techniques and Applications, J. Hurrel (CRC Press, 1982)). Furthermore, a general review of techniques concerning the synthesis of antibody fragments that retain the specificity of the native antibody bond can be found in Winter et al. (1991, Nature 349, 293-299). Some antibodies capable of bonding it to CSF-1 or its receptor are commercially available (for example from Oncogene Science, Manhasset, N.Y., USA). For example, there is the monoclonal antibody described by Birchenall-Roberts et al. (J. Immunol, 1990, 145, 3290-3296).

According to another embodiment of the invention, the substance capable of inhibiting the bond of CSF-1 to its receptor is a peptide capable of bonding to CSF-1 or to the CSF-1 receptor. These peptides may be prepared according to procedures available in prior art. For example, these peptides may be prepared by Phage display techniques (Smith, G. P., Curr. Opin. Biotechnol. 2:668-673 (1991)).

“Substance with at least one cytotoxic activity” implies that the substance considered is capable of inducing or activating an immune response directed specifically against a target cell or inhibiting the growth and/or division of such a cell. According to one preferred case, this cytotoxic activity results in the death of the said cell. In one preferred embodiment of the invention, the said target cell is a tumour cell (the cytotoxic activity is then called an anti-tumour activity).

The cytotoxic activity of a given substance, and particularly an anti-tumour substance, may be evaluated in vitro by measuring cell survival, ideally by short term viability tests (for example such as the tryptan blue or MTT test), or by cionogenic survival tests (formation of colonies) (Brown and Wouters, 1999, Cancer Research, 59, 1391-1399) or in vivo by measurement of the growth of tumours (size and/or volume) in an animal model (Ovejera and Houchens, 1981, Semin. Oncol., 8, 386-393).

According to one embodiment of the invention, the substance with at least one cytotoxic activity is chosen from among substances interacting with DNA, antimetabolites, topo-isomerase inhibitors and spindle agents.

Substances interacting with DNA are substances currently used for anti-cancer treatments. In particular they include alkylants, methyl donors (for example nitroso-ureas), organoplatines (particularly cisplatine, carboplatine and oxaliplatine) and DNA splitting agents.

Antimetabolites act by inhibiting cellular metabolism and are also well known to those skilled in the art. In particular, there are the antifolics, for example such as methotrexate and raltitrexed, antipurics such as cladribine, fludarabine, mercaptopurine and pentostatin, and antipyrimidics, for example such as cytarabine, Fluorouracil, and Gemcitabine.

Topo-isomerase inhibitors include particularly topo-isomerase inhibitors I (e.g. irinotecan and topotecan), topo-isomerase II inhibitors (e.g. anthracyclines).

Spindle agents are substances that block the formation or disappearance of the mitotic spindle thus inhibiting cellular mitosis. These include vincristine, vinblastine and taxoids.

According to one preferred embodiment of the invention, the substance with at least one cytotoxic activity is chosen from among antigens associated with tumours (AAT). Some tumour cells express specific antigens. These AATs may be used for the context of an immunotherapeutic treatment, to initiate an immuno-response against tumour cells (for example, there is a review in Bacchia et al. Haematologica, 2000, 85, 1172-206).

AATs that can be used in the context of the invention include but are not limited to MART-1 (Kawakami et al. J. Exp. Med. 180:347′-352, 1994) MAGE-1, MAGE-3, GP-100, (Kawakami et al. Proc. Nat. Acad. Sci. U.S.A. 91:6458-6462, 1994), CEA, tyrosinase (Brichard et al. J. Exp. Med. 178:489, 1993), MUC-1, MUC-2, mutated ras oncogene, oncogene p53, CA-125, PSA, PSMA, PAP and c-erb/B2 (Boon et al., Ann. Rev. Immunol, 12;337, 1994).

According to another embodiment of the invention, the substance with at least one cytotoxic activity is chosen from among cytokines, polypeptides with a chemo-attraction activity (i.e. chemokines), proteins coded by a gene called a “suicide gene”, anti-angiogenic proteic factors and polypeptides with a cellular apoptosis activation activity.

Cytokines are molecules produced naturally following an antigenic stimulation or an inflammatory reaction (Gillis and Williams, 1998, Curr. Opin. Immunol., 10, 501-503), for which the usefulness in the treatment of some cancers has been demonstrated particularly by Oettger (Curr. Opin. Immunol., 1991, 3, 699-705). According to this first variant of the invention, the substance with at least one cytotoxic activity will preferably denote a cytokine chosen from among α, β and γ interferons, interleukins, and particularly IL-2, IL-4, IL-6, IL-10 or IL-12, tumour necrosing factors (TNF) and factors stimulating colonies (for example GM-CSF, C-CSF, M-CSF).

According to one preferred embodiment, the said cytokine is selected from among interleukin-2 (IL-2) and gamma interferon (IFN-γ). In particular, interleukin-2 is responsible for the proliferation of activated T lymphocytes, multiplication and activation of cells of the immune system (in particular, see EP0206939 for the nucleic acid sequence). IFN-γ activates phagocyte cells and increases the expression of class I or class II surface antigens in the major histocompatibility complex (in particular, see EP020620 for the nucleic acid sequence). These nucleic acid sequences are incorporated into the application by reference.

According to one particular embodiment, the combination product according to the invention is characterised in that it comprises at least two substances with at least one cytotoxic activity, namely interleukin-2 (IL-2) and gamma interferon (IFN-γ).

According to another variant of the invention, the substance with at least one cytotoxic activity is a polypeptide with a chemo-attraction activity (i.e. chemokines). Chemokines form a sub-class of the cytokines family. They are distinguished from other cytokines by their chemo-attractive property, particularly during natural chemotactism processes, and particularly the attraction of cells in the immune system towards tissues in which the inflammation or infection is located, and by their anti-angiogenic properties.

Chemokines are proteins with low molecular weight (between 8 and 10 kd), and are small (from 70 to 80 amino acids), and their amino acid sequences have a low homology ratio (varying from 10 to 70% depending on the chemokines considered), so that about. 50 different chemokines have been defined so far. Nevertheless, these chemokines have been subdivided into four large families related to the position of the cystein residues that they enclose. The α family for which the N-terminal end comprises 2 cysteins separated by a single amino acid (type IL-8, NAP-2, GCP-2 chemokines) and the ∃ family for which the N-terminal end comprises 2 adjacent cysteins (RANTES, MIP1, MCP1 type chemokines) are the best characterised (Horuk, R., 1994, Trends Pharmacol, Sci., 15, pages 159-165; Murphy, P. M., 1994, Annu. Rev. Immunol., 12, pages 593-633).

In the context of this invention, the preferred chemokine is chemokine type MIP 1, and more particularly selected among the group consisting of chemokines MIP1α and MIP1β for which the properties have been demonstrated by Wolpe et al (1988, J. Exp. Med, 167, 570-581).

MIP1α, for which nucleic and peptidic acid sequences are described in Obaru et al. (1986, J. Biochem., 99, 885-894), the content of which is incorporated into this application by reference, is produced by T lymphocytes and monocytes. It enables chemo-attraction of eosinophiles and T lymphocytes during infections of the respiratory tracts; monocytes and neutrophiles during rheumatoidal arthritis, inflammations of the digestive system or meningitis with bacterial origin. It also inhibits proliferation of hematopoietic precursors.

MIP1β, for which the nucleic and peptidic acid sequences are described in Brown et al. (1989, J. Immunol, 142, 679-68), the content of which is incorporated into this application by reference, is also produced by T lymphocytes and monocytes. It exerts its chemo-attractive properties on monocytes and neutrophiles in cases of bone arthritis and bacterial meningitis. Like MIP1α, it inhibits the proliferation of hematopoietic precursors.

There are natural variants of the said MIP1α and MIP1β proteins known to those skilled in the art, for example called GOS19, LD78, pAT464, TY5 (mouse) or SIS α (mouse) for MIP1α or pAT744, Act-2, G-26n H-400 (mouse) or hSIS γ (mouse) for MIP1β. In the special case of MIP1β, for example the sequence corresponding to Act-2 (Lipes et al., 1988, PNAS, 85, 9704-9708) may be chosen, the content of which is incorporated here by reference).

According to another variant of the invention, the substance with at least one cytotoxic activity is a polypeptide coded by a gene called a “suicide gene”. Several studies have identified polypeptides that are not toxic as such but that have catalytic enzymatic properties capable of transforming an inactive substance (pre-drug), for example a nucleoside or a nucleoside analogue, into a highly toxic substance for the cell, for example a modified nucleoside that can be incorporated into DNA or RNA chains in elongation, particularly with the consequence of inhibiting cellular division or cellular malfunctions leading to death of the cell containing such polypeptides. Genes coding for such polypeptides are said to be “suicide genes”. Many suicide genes/pre-drug pairs are now available. For example, there are the following pairs:
herpes simplex virus type 1 thymidine kinase (TK-HSV-1) and acyclovir or ganciclovir (GCV) (Caruso et al., 1993, Proc. Natl. Acad. Sci. USA 90, 7024-7028; Culver et al., 1992, Science 256, 1550-1552; Ram et al., 1997, Nat. Med. 3, 1354-1361);
rat cytochrom p450 and cyclophosphophamide (Wei et al. 1994, Human Gene Therapy 5, 969-978);
purine nucleoside phosphorylase of Escherichia coli (E. Coli) and 6-methylpurine deoxyribonucleoside (Sorscher et al., 1994, Gene Therapy 1, 233-238);
guanine phosphoribosyl transferase of E. Coli and 6-thioxanthine (Mzoz et Moolten, 1993, Human Gene Therapy 4, 589-595) and
cytosine deaminase (CDase) and 5-fluorocytosine (5FC).

According to one advantageous case, the invention relates to the case in which the said substance with at least one cytotoxic activity has at least one enzymatic activity selected from among the thymidine kinase activity, the purine nucleoside phosphorylase activity, guanine or uracil or orotate phosphoribosyl transferase activity and cytosine deaminase activity.

More particularly CDase is an enzyme that acts in the metabolic channel of pyrimidines, according to which exogenic cytosine is transformed through a hydrolytic deamination in uracil. CDase activities have been demonstrated in prokaryotes and lower eukaryotes (Jund and Lacroute, 1970, J. Bacteriol. 102, 607-615; Beck et al., 1972, J. Bacteriol. 110, 219-228; De Haan et al., 1972, Antonie van Leeuwenhoek 38, 257-263; Hoeprich et al., 1974, J. Inf. Dis. 130, 112-118; Esders and Lynn, 1985, J. Biol. Chem. 260, 3915-3922) but they are absent in mammals (Koechlin et al., 1966, Biochem Parmacol. 15, 435-446; Polak et al., 1976, Chemotherapy 22, 137-153). FCY1 genes of Saccharomyces cerevisiae (S. cerevisiae) and codA of E. coli coding for CDase of these two organisms respectively are known and their sequences have been published (EP 402 108; Erbs et al., 1997, Curr. Genet. 31, 1-6;

WO93/01281). CDase also deamines an analogue of cytosine, 5-fluorocytosine (5-FC) in 5-fluorouracil (5-FU) which is a highly cytotoxic compound particularly when it is converted into 5-fluoro-UMP (5-FUMP). Cells with no CDase activity, either due to inactivating mutation of the gene coding for the enzyme, or due to their natural deficiency for this enzyme (for example mammal cells) are resistant to 5-FC (Jund and Lacroute, 1970, J. Bacteriol. 102, 607-615; Kilstrup et al., 1989, J. Bacteriol., 171, 2124-2127). On the other hand, it has been shown that the sensitivity to 5-FC in mammal cells in which the sequence coding for a CDase activity has been transferred (Huber et al., 1993, Cancer Res. 53, 4619-4626; Mullen et al., 1992, Proc. Nati. Acad. Sci. USA 89, 33-37; WO 93/01281) can be transmitted. Moreover, in this case, the bystander cells not transformed also become sensitive to 5-FC (Huber et al., 1994, Proc. Natl. Acad. Sci. USA 91, 8302-8306). This phenomenon, called the bystander effect, is due to excretion by cells expressing the CDase activity, of 5-Fu that intoxicates bystander cells by simple diffusion through the cellular membrane. This passive diffusion property of 5-FU is an advantage over the tk/GCV reference system for which the bystander effect requires contact with the cells that express tk (Mesnil et al., 1996, Proc. Natl. Acad. Sci. USA 93, 1831-1835). Consequently, this effect is an additional advantage in the use of CDase for genetic therapy particularly anti-cancer therapy.

However, the sensitivity to 5-FC varies very much depending on the cell lines. For example low sensitivity is observed in human tumour lines PANC-1. (carcinoma of pancreas) and SK-BR-3 (adenocarcinoma of the breast) transduced by a retrovirus expressing the coda gene of E. coli (Harris et al., 1994, Gene Therapy 1, 170-175). This undesirable phenomenon could be explained by the absence or the low endogenic conversion of 5-FU formed by the enzymatic action of CDase into cytotoxic 5-FUMP. This step, normally performed in mammal cells by orotate phosphorybosyl transferase (Peters et al., 1991, Cancer 68, 1903-1909), may be missing in some tumours and thus make genic therapy based on CDase inoperative. In lower prokaryotes and eukaryotes, uracil is transformed into UMP by the action of uracil phosphoribosyl transferase (consequently presenting a UPRTase activity). This enzyme also converts 5-FU into 5-FUMP. Thus furl mutants of yeast S. cerevisiae are resistant to high concentrations of 5-FU (10 mM) and 5-FC (10 mM), because in the absence of any UPRTase activity, the 5-Fu originating from deamination of 5-FC by CDase is not transformed into a cytotoxic 5-FUMP (Jund and Lacroute, 1970, J. Bacteriol. 102, 607-615). The upp and FUR1 genes coding for UPRTase of E. coli and S. cerevisiae respectively, were cloned and sequenced (Andersen et al., 1992, Eur. J. Biochem. 204, 51-56; Kern et al., 1990, Gene 88, 149-157).

According to one embodiment of this invention, the substance with at least one cytotoxic activity has a UPRTase activity, which means that the said polypeptide is capable of converting uracil or one of its derivatives into a monophosphate analogue, and particularly 5-FU into 5-FUMP.

The UPRTase with which this invention is concerned may have any origin, particularly prokaryotic, fungal or yeast. For example, nucleic acid sequences coding for E. coli (Anderson et al., 1992, Eur. J. Biochem 204, 51-56), Lactococcus lactis (Martinussen and Hammer, 1994, J; Bacteriol. 176, 6457-6463), Mycobacterium bovis (Kim et al., Biochem Mol. Biol. Int 41, 1117-1124) and Bacillus subtilis (Martinussen et al., 1995, J. Bacteriol. 177, 271-274) UPRTases, may be used in the context of the invention. But in particular, it is preferred to use a yeast UPRTase and particularly that coded by the FUR1 gene of S. cerevisiae, for which the sequence divulged in Kern et al. (1990, Gene 88, 149-157) is introduced here by reference. As guidance, the sequences of genes and the sequences of corresponding UPRTases may be found in the literature and specialised databanks (SWISSPROT, EMBL, Genbank, Medline, etc.).

Furthermore, application WO9954481 describes a FUR1 gene deprived of 105 nucleotides in 5′ of the coding part enabling synthesis of a UPRTase deleted from the first 35 residues in the N-terminal position and beginning with methionine in position 36 in the native protein. The expression product of the mutating gene called FUR1)105, is capable of complementing a furl mutant of S. cerevisiae. On the other hand, the truncated mutant has a UPRTase activity greater than the native enzyme. Thus, according to one particularly advantageous embodiment, the polypeptide coded according to the invention is a deletion mutant of a native UPRTase. The deletion is preferably located in the N-terminal region of the original UPRTase. It may be total (concern all residues of the said N-terminal region) or partial (concern one or several continuous or non continuous residues in the primary structure). In general, a polypeptide is composed of N-terminal, central and C-terminal parts, each representing about one third of the molecule. For example, UPRTase of S. cerevisiae with 251 amino acids, its N-terminal part is composed of the 83 first residues beginning with the so-called initiating methionine located in the first position of the native form. As for UPRTase of E. coli, its N-terminal part covers positions 1 to 69.

Furthermore, patent applications WO96/16183 and WO99554481 describe the use of a fusion protein coding for an enzyme with two domains with CDase and UPRTase activities and demonstrating that the transfer of a hybrid gene codA::upp or FCY1::FUR1 or FCY1::FUR1Δ105 carried by an expression plasmide increases the sensitivity of transfected B16 cells to 5-FC. The proteic and nucleic sequences described in these two applications are incorporated into the description of this application. According to this embodiment, the polypeptide is a polypeptide fused in phase with at least one second polypeptide. Although the fusion can take place at any location on the first polypeptide, the N or C-terminal ends are preferred and particularly the N-terminal end. Fusion of the CDase and UPRTase activities provides a means of improving the sensitivity of target cells to 5-FC and 5-FU.

Those skilled in the art are capable of cloning CDase or UPRTase sequences from published data, making mutations if necessary, testing enzymatic activities of mutating forms in a non-cellular or cellular system according to the state of the art, or following the protocol indicated in application WO9954481 and fusioning CDase and UPRTase activity polypeptides, particularly in phase, and consequently all or some of the corresponding genes. Hybrid polypeptides such as those described in patent applications WO96/16183 and WO9954481 are incorporated into this application by reference.

According to another variant, the substance with at least one cytotoxic activity is an anti-angiogenic proteic factor. Angiogenesis is the process responsible for the formation of new capillaries from an existing vascular network. This complex process is finely regulated in healthy tissues by the balance of the effects of many angiogenic and anti-angiogenic factors. However, this process is deregulated in some pathologies, and particularly in the formation of a tumour; the angiogenic factors are stronger than anti-angiogenic factors which enables large vascularisation of tumours and consequently their fast development and/or the appearance of metastases. This is why, in the context of this invention, an anti-angiogenic factor is considered as being a cytotoxic agent, particularly an anti-tumour agent. The various anti-angiogenic factors known at the present time include angiostatin, endostatin, the platelet factor 4 (PF4), thrombospondin-1, PRP (Proliferin Related Protein), VEGI (Vascular Endothelial Growth Inhibitor), metalloproteases and urokinase.

According to another variant of the invention, the substance with at least one cytotoxic activity may be a polypeptide presenting an activity to activate cellular apoptosis, and particularly protein p53. p53 is a nuclear phosphoprotein involved particularly in control of the expression of the proteins involved in the cellular cycle (Ozbun et al., 1995, Adv. Cancer Res. 66, 71-141—Selter et al., 1994, Int. J. Biochem. 26, 145-154) and participating in a large number of cellular processes related to stability of the genome and cellular apoptosis (Harris et al, 1996, J. Natl. Cancer Inst. 88, 1442-1445; Kastan et al, 1991, Cancer Res. 51, 6304-6311; Kuerbitz et al., 1992, PNAS, 89, 7491-7495). The p53 gene has been identified and sequenced. The cDNA sequence is described in Matlashewski et al. (1984, EMBO J., 3, 3257-3262) and the protein sequence has been described in Lamp (1986, Mol. Cell Biol., 6, 1379-1385). Similarly, natural and functional polymorphic variants have been identified for which some amino acids are replaced by others, without affecting the p53 function. Moreover, a large number of mutations have been described in the literature related to cancer that can result in the loss of function of this protein (Holstein et al., 1991, Science, 253, 49-53; Levine et al., 1991, Nature, 351, 453-456). For example, Baker et al., (1989, Science, 244, 217) have observed that the function of this p53 gene is lost in more than 70% of colorectal tumours. In the context of this invention, the entire nucleic acid sequence coding for the p53 polypeptide may be used, or only part of this polypeptide or a derived or mutated polypeptide may be used, provided that the p53 function is conserved. This type of sequence is well known to those skilled in the art and for example reference can be made to Matlashewski et al. (1984, EMBO J., 3, 3227-3262), Prives et al. (1994, Cell, 78, 543-546) or Chen et al. (1996, Gene and Deve, 10, 2438-2451), the contents of which are incorporated into this application. Considering the properties of the p53 polypeptide as a transcriptional transactivator (Farmer et al., 1992, Nature, 358, 83-86) or as a polypeptide capable of interacting with other proteins (Harris, 1996, Carcinogenesis, 17, 1187-1198), the p53 activity may be measured by analysis of the stoppage of the cell cycle in the G1/S and G2/M phases, induction of apoptosis, suppression of cell transformation induced by oncogenes or inhibition of angiogenesis.

According to one particular embodiment of the invention, the said combination process also comprises pharmaceutically acceptable quantities of a prodrug that can be transformed into a cytotoxic molecule by a substance with at least one cytotoxic activity. In particular, this type of prodrug will be selected from the group consisting of acyclovir or ganciclovir (GCV), cyclophosphophamide, 6-methylpurine deoxyribonucleoside, 6-thioxanthine, cytosine or one of its derivatives or uracil or one of its derivatives. Moreover, when the said prodrug is 5-fluorocytosine (5FC) or 5-fluorouracil (5-FU), the said combination product may also include one or several substances potentialising the cytotoxic-effect of 5-FU. In particular, there are drugs inhibiting enzymes of the novo pyrimidine biosynthesis method (for example see those mentioned below), drugs such as Leucovorin (Waxman et al., 1982, Eur. J. Cancer Cin. Oncol. 18, 685-692) that in the presence of the 5-FU metabolism product (5-FdUMP) increases inhibition of thymidylate synthase which causes a reduction in the pool of dTMP necessary for replication and finally drugs such as methotrexate (Cadman et al., 1979, Science 250, 1135-1137) that inhibits dihydrofolate reductase and increases the incorporation pool of PRPP (phosphoribosylpyrophosphate) to increase 5-FU in cell RNA.

“Nucleic acid” means a double strand or single strand, linear or circular, natural isolated or synthetic fragment of DNA and/or RNA, denoting a precise sequence of nucleotides, modified or not modified, used to define a fragment or a region of a nucleic acid without any size limitation.

According to one preferred embodiment of the invention, the nucleic acid according to the invention will be a vector. Preferably, according to this invention, “nucleic acid” denotes a recombining vector of plasmidic or viral origin. The choice of plasmides that can be used in the context of this invention is enormous. It may include cloning and/or expression vectors. In general, they are known to those skilled in the art and many of them are commercially available, but they can also be constructed or modified using genetic manipulation techniques. For example, there are plasmides derived from pBR322 (Gibco BRL), pUC (Gibco BRL), pBluescript (Stratagene), pREP4, pCEP4 (Invitrogene) or p Poly (Lathe et al., 1987, Gene 57, 793-201). Preferably, a plasmide used in the context of this invention contains a replication origin initiating the replication in a producing cell and/or a host cell (for example, the origin ColE1 will be used for a plasmide to be produced in E. coli and the oriP/EBNA1 system will be used if it is required that it should be auto-replicative in a mammal host cell (Lupton and Levine, 1985, Mol. Cell. Biol. 5, 2533-2542; Yates et al., Nature 313, 812-815). It may also include a selection gene for selecting or identifying transfected cells (for example complementation of an auxotrophy mutation, gene coding for resistance to an antibiotic). Obviously, it may include supplementary elements improving its maintenance and/or stability in a given cell (cer sequence that facilitates maintaining a plasmide in monomer form) (Summers and Sherrat, 1984, Cell 36, 1097-1103), integration sequences and the cellular genome.

Since it is a viral vector, a vector deriving from a poxvirus could be envisaged (for example the vaccine virus, particularly MVA, canaripox), an adenovirus, a retrovirus, a herpes virus, an alphavirus, a foamy virus or a virus associated with adenovirus. Preferably, a non-replicative and non-integrative vector will be used. In this respect, adenoviral vectors are perfectly suitable for implementation of this invention. However, note that the nature of the vector is not very important when considering implementation of this invention.

Retroviruses have the property that most of them infect and integrate into cells in division and in this respect are particularly appropriate for a cancer application. A recombining retrovirus according to the invention generally includes LTR sequences, a packaging region and the nucleotidic sequence according to the invention placed under the control of retroviral LTR or an internal promoter like those described below. It may be derived from a retrovirus from any origin (murine, primate, feline, human, etc.) and particularly MoMulV (Moloney murine leukaemia virus), MVS (Murine sarcoma virus) or Friend murine retrovirus (Fb29). It is propagated in a packaging line capable of supplying gag, pol and/or env viral polypeptides necessary for the constitution of a viral particle, in trans. This type of line is described in the literature (PA317, Psi CRIP GP+Am-12, etc.). The retroviral vector according to the invention may include modifications particularly at LTRs (replacement of the promoting region by a eukaryote promoter) or the packaging region (replacement by a heterologous packaging region, for example of the type VL30) (see applications WO9601324 and FR2762625).

A defective adenoviral vector could also be used for replication, in other words deprived of all or part of at least one region essential for replication selected among the E1, E2, E4 and/or L1-L5 regions. Deletion of the E1 region is preferred. But it may be combined with other modifications/deletions, particularly affecting all or part of the E2, E4 and/or L1-L5 regions, provided that the defective essential functions are complemented in trans by means of a complementation line and/or an auxiliary virus in order to assure production of the viral particles of interest. In this respect, vectors according to prior art can be used for example such as those described in international applications WO 94/28152 and WO 97/04119. For illustration, deletion of the majority of region E1 and the transcription unit E4 is particularly advantageous. In order to increase cloning capabilities, the adenoviral vector may also be deprived of all or part of the non-essential E3 region. According to another alternative, a minimum adenoviral vector could be used retaining only the essential sequences for packaging, namely ITRs (Inverted Terminal Repeat), 5′ and 3′ and the packaging region. Moreover, the origin of the adenoviral vector according to the invention may be varied both in terms of the species and the serotype. It may be derived from the genome of an adenovirus with human or animal origin (for example canine, avian, bovine, murine, ovine, porcine, simian) or from a hybrid comprising fragments-of the adenoviral genome of at least two different origins. More particularly, it is worth mentioning the CAV-1 or CAV-2 adenoviruses with canine origin, DAV with avian origin and type 3 Bad with bovine origin (Zakharchuk et al., Arch. Virol., 1993, 128: 171-176; Spibey and Cavanagh, J. Gen. Virol., 1989, 70: 165-172; Jouvenne et al., Gene, 1987, 60: 21-28; Mittal et al., J. Gen Virol., 1995,-76: 93-102). However, an adenoviral vector with human origin will be preferred, preferably deriving from a serotype C adenovirus, particularly type 2 or 5. An adenoviral vector according to this invention may be generated in vitro in Escherichia coli (E. coli) by ligation or homologous recombination (for example see international application WO 96/17070) or by recombination inca complementation line. The different adenoviral vectors and their preparation techniques are known (for example see Graham and Prevect, 1991, in Methods in Molecular Biology, vol. 7, p 109-128; Ed: E. J. Murey, The Human Press Inc).

If the nucleic acid comprises at least one sequence coding an oligonucleotide and/or a polypeptide, it should be mentioned that the said nucleic acid also comprises, elements necessary to assure expression of the said sequence after transfer into a target cell, particularly of promoting sequences and/or regulation sequences efficient in the said cell, and possibly the required sequences to enable excretion or expression on the surface of the target cells of the said polypeptide. Elements necessary for the expression include all elements enabling transcription of the nucleotidic sequence in RNA and translation of mRNA into polypeptide, particularly promoting sequences and/or regulation sequences efficient in the said cell and possibly sequences required to enable excretion or expression on the surface of the target cells of the said polypeptide. These elements can be regulatable or constitutive. Obviously, the promoter is adapted to the selected vector and to the host cell. For example, it is worth mentioning eukaryote promoters of PGK (Phospho Glycerate Kinase) genes, MT (metallothioneine; Mc Ivor et al., 1987, Mol. Cell Biol., 7, 838-848), α-1 antitrypsine, CFTR, gene promoters coding for muscular creatine kinase, for actin, for pulmonary surfactant, for immunoglobulins, for β-actin (Tabin et al., 1982, Mol. Cell Biol., 2, 426-436), SRα (Takebe et al., 1988, Mol. Cell. Biol., 8, 466-472), the precocious promoter of the SV40 virus (Simian virus), the LTR or RSV (Rous Sarcoma Virus), the MPSV promoter, the promoter TK-HSV-1, the precocious promoter of the CMV (Cytomegalovirus) virus, promoters of the virus of vaccine p7.5K pH5R, pK1L, p28, p11 and adenoviral promoters E1A and MLP or a combination of the said promoters. It may also be a promoter stimulating the expression of the gene in a tumour cell. For example, it is worth noting particularly promoters of MUC-1 genes overexpressed in cancers of the breast and the prostate (Chen et al., 1995, J. Clin. Invest., 96, 2775-2782), CEA (carcinoma embryonic antigen) overexpressed in cancers of the colon (Schrewe et al., 1990, Mol. Cell. Biol., 10, 2738-2748), tyrosinaemia overexpressed in melanomas (Vile et al., 1993, Cancer Res. 53, 3860-3864), ERB-2 overexpressed in cancers of the breast and-pancreas. (Harris et al., 1994, Gene Therapy, 1, 170-175) and α-fetoprotein overexpressed in cancers of the liver (kanai et al., 1997, Cancer Res., 57, 461-465). The precocious promoter of Cytomegalovirus (CMV) is preferred in particular. It is also possible to use a tissue specific promoting region, particularly when the tumour to be treated is derived from a particular cell type, or can be activated under defined conditions. The literature provides a great deal of information about this type of promoting sequence. Similarly, “neutral” nucleic sequences or introns can be introduced in this type of nucleic acid construction that do not harm the transcription and are spliced before the translation step. This type of sequence and their uses are described in the literature (WO 94/29471). The said nucleic acid may also contain sequences required for intracellular transport, for replication and/or integration, for secretion, for transcription or translation. This type of sequence is well known to those skilled in the art. Moreover, nucleic acids that can be used according to this invention may also be nucleic acids modified such that it is impossible for them to be integrated into the genome of the target cell or nucleic acids stabilised using agents, for example such as spermine, which as such have not effect on the efficiency of transfection.

According to one preferred variant of the invention, the combination product according to the invention comprises at least one nucleic acid comprising at least one sequence coding for a substance capable of inhibiting the CSF-1 activity and at least one sequence coding for a substance with at lest one cytotoxic activity.

In the context of this invention, it is possible to use all or only part of the sequence of nucleic acid coding for a substance capable of inhibiting the CSF-1 activity or for a substance with at least one cytotoxic activity, or a derived or mutated substance, provided that the functions or properties of these substances are preserved. For the purposes of this invention, mutation means deletion and/or substitution and/or addition of one or several nucleotides. Similarly, it would be possible to use a sequence coding for a hybrid polypeptide originating from fusion of the sequence coding for a substance capable of inhibiting the activity of CSF-1 or for a substance with at least one cytotoxic activity and the sequence coding for a polypeptide of another type (for example membrane anchorage, secretion).

Nucleic acids and oligonucleotides according to the invention may advantageously be associated with at least one substance that associates with nucleic acids.

“Substance that associates with nucleic acids” means a substance, or a combination of several substances, which can in particular improve the transfectional efficiency and/or the stability of a vector and/or an oligonucleotide, particularly a vector with plasmidic origin, and/or protection of the said vector or the said oligonucleotide in vivo with regard to the immune system of the host organism (Rolland A., Critical reviews in Therapeutic Drug Carrier System, 15, (1998), 143-198). These substances can be associated with nucleic acids by electrostatic, hydrophobic, cationic, covalent interactions, or preferably non-covalent interaction. These types of substances are largely documented in the literature accessible to those skilled in the art (for example see Felgner et al., 1987, Proc. West. Pharmacol. Soc. 32, 115-121; Hodgson and Solaiman, 1996, Nature Biotechnology 14, 339-342; Remy et al., 1994, Bioconjugate Chemistry 5, 647-654). For illustration, but in no way limitatively, they may be cationic polymers, cationic lipids, but also liposomes, nuclear or viral proteins or neutral, zwitterionic or negatively charged lipids. These substances may be used alone or in combination. Examples of such compounds, and methods of measuring their capacity to improve the transfectional efficiency and/or stability of a given vector, are available particularly in patent applications WO 98/08489, WO 98/17693, WO 98/34910, WO 98/37916, WO 98/53853, EP 890362 and WO 99/05183. In particular, it may consist of lipidic substances such as DOTMA (Felgner et al., 1987, PNAS, 84, 7413-7417), DOGS or Transfectam™ (Behr et al., 1989, PNAS, 86, 6982-6986), DMRIE or DORIE (Felgner et al., 1993, Methods, 5, 67-75), DC-CHOL (Gao and Huang, 1991, BBRC, 179, 280-285), DOTAP™ (McLachlan et al., 1995, Gene Therapy, 2, 674-622) or Lipofectamine™.

Advantageously, these cationic lipids are selected from among cationic lipids with the following formula (see EP 901 463): 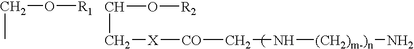
in which:
R₁, R₂ may be identical or different and are linear or ramified C₆-C₂₃ alkyls or C₆-C₂₃ alkenyls, or linear or ramified C₆-C₂₃ alkyl carbonyls or C₆-C₂₃ alkenyl carbonyls.
X is O, S, S(O) or —NR₃, R3, are atoms of hydrogen or C₁-C₄ alkyls,
n is a positive integer number between 1 and 6,
m is a positive integer number between 1 and 6, and when n>1, m can vary within the same molecule.

The substance that is associated with nucleic acids may also be a cationic polymer, for example such as polyamidoamine (Haensler and Szoka, Bioconjugate Chem. 4 (1993), 372-379), a dendrimer polymer (WO 95/24221), polyethylene imine or polypropylene imine (WO 96/02655), chitosan, poly(aminoacid) such as polylysine (U.S. Pat. No. 5,595,897 or FR 2 719 316); a polyquaternary compound; protamine; polyimines; polyethylene imine or polypropylene imine (WO 96/02655); polyvinylamines; polycationic polymers substituted by DEAE such as pullulans, celluloses; polyvinylpyridine; polymethacrylates; polyacrylates; polyoxethanes; polythiodiethylaminomethylethylene (P(TDAE)); polyhistidine; polyornithine; poly-p-aminostyrene; polyoxethanes; copolymethacrylates (for example HPMA copolymers; N-(2-hydroxypropyl)-methacrylamide); compounds described in U.S. Pat. No. 3,910,862, polyvinylpyrrolide complexes of DEAE with methacrylate, dextran, acrylamide, polyimines, albumin, 1-dimethylaminomethylmethacrylate and ammonium chloride of polyvinylpyrrolidonemethylacrylaminopropyltrimethyl; polyamidoamines; telomeric compounds (patent application EP0965583). Nevertheless, this list is not exhaustive and other known cationic polymers could be used to obtain complexes of nucleic acids according to the invention. Moreover, these cationic lipids and polymers may be fluorinated (for example see WO 98/34910).

The substance that is associated with nucleic acids may also be a neutral, zwitterionic or negatively charged lipid. These neutral, zwitterionic or negatively charged lipids may for example be selected from the group comprising natural phospholipids with animal or vegetable origin, such as phosphatidylcholine, phosphocholine, phosphatidylethanolamine, sphingomyeline, phosphatidylserine, phosphatidylinositol, ceramid or cerebroside and their analogues; synthetic phospholipids that usually but not exclusively comprise two identical fatty acid chains such as dimyristoylphosphatidylcholine, dioleoylphosphatidylcholine, dipalmitoylphosphatidylcholine, distearoylphosphatidylcholine, phosphatidylethanolamine (PE) and phosphatidylglycerol and their analogues; phosphatidylcholine, cardiolipin, phosphatidylethanolamine, mono-, di-, or triacylglycerol, and alpha-tocopherol and their analogues; phosphatidylglycerol, phosphatidic acid or analogue of a similar phospholipid; cholesterol, glycolipids, fatty acids, sphingolipids, prostaglandins, gangliosides, niosomes, or any other natural or synthetic amphiphile.

Moreover, oligonucleotides and nucleic acids used in the context of this invention may also include targeting elements that can direct the transfer of the said nucleic acid sequence(s) to some cell types or some particular tissues (tumour cells, pulmonary epithelium cells, haematopoietic cell, muscle cell, nerve cell, etc.). They can also enable redirection of the transfer of an oligonucleotide or a nucleic acid to some preferred intracell compartments such as the nucleus and mitochondrias. They may also use elements facilitating penetration inside the cell or the lyse of the endosomes. This type of targeting element is broadly described in the literature. For example, it may be all or part of lectins, peptides, particularly JTS-1 peptide (see patent application WO 94/40958), oligonucleotides, lipids, hormones, vitamins, antigenes, antibodies, specific ligands of membrane receptors, ligands that can react-with an anti-ligand, fusogenic peptides, nuclear localisation peptides, or a combination of such compounds. In particular, it may be galactosyl residues used to target the receptor of asialoglycoproteins on the surface of hepatic cells, ligands capable of interacting with receptors such as growth factor receptors, cytokine, lectin, adhesion protein receptors, it may also be an antibody fragment such as the Fab fragment, a fusogenic peptide INF-7 derived from the sub-unit HA-2 of hemagglutinin of the influenza virus (Plank et al., 1994, J. Biol. Chem. 269, 12918-12924), a nuclear localisation signal derived from the T antigen of the SV40 virus or the EBNA-1 protein of the Epstein Barr virus.

The invention also relates to a pharmaceutical composition characterised in that it comprises an oligonucleotide capable of inhibiting the expression of CSF-1 according to the invention and/or a nucleic acid comprising a sequence coding for such an oligonucleotide and a pharmaceutically acceptable vehicle. The invention also relates to a pharmaceutical composition characterised in that it comprises a combination product like that previously described and a pharmaceutically acceptable vehicle. This type of support is preferably isotonic, hypotonic or slightly hypertonic, and has a relatively weak ionic force, for example such as a sucrose solution. Furthermore, this type of support may contain any solvent, or aqueous or partially aqueous liquid such as sterile non-pyrogenic water. The pH of the formulation is also adjusted and buffered so as to satisfy in vivo usage requirements. The formulation may also include a diluent, an additive or a pharmaceutically acceptable excipient, in the same way as solubilisation, stabilisation and preservation agents. For an injectable administration, it is preferred to use a formulation in an aqueous, non-aqueous or isotonic solution. It may be presented in a single dose or in multi-doses, in liquid or dry form (powder, freeze dried, etc.) that can be extemporaneously reconstituted by an appropriate diluent.

The invention also relates to a method of inhibiting the expression of CSF-1 consisting of bringing a cell that expresses CSF-1 into contact with an oligonucleotide according to the invention and/or with a nucleic acid comprising a sequence coding for such an oligonucleotide.

Another purpose of the invention is to use an oligonucleotide capable of inhibiting the expression of CSF-1, or a nucleic acid comprising a sequence coding for such an oligonucleotide for preparation of a drug intended for the treatment of the human or animal body, intended particularly for cancer treatment.

The invention also relates to the use of a substance capable of inhibiting the activity of CSF-1 for the preparation of a drug to improve the efficiency of an anti-tumour treatment.

The most appropriate doses vary as a function of the different parameters, for example such as the individual or the disease to be treated or the administration method, or the host organ/tissue. For instance, oligonucleotide doses vary from 0.6 to 6.5 mg/kg/day.

Another purpose of the invention is to use a recombination product like that described above for the preparation of a drug intended for the treatment of a human or animal body, particularly intended for cancer treatment.

The combination product according to the invention may be used in the context of simultaneous, consecutive or spread over time use. Simultaneously means co-administration. In this case, compounds (i) and (ii) may be mixed before administration or may be administered at the same time in the host cell or organism. They may also be administered consecutively, in other words one after the other, regardless of which component of the combination product according to the invention is administered first. Finally, an administration method spread over time or intermittent method is possible in which one and/or the other of the two components may be stopped and resumed at regular or irregular intervals. Note that the methods and administration sites of the two components may be different.

According to one preferred embodiment of the invention, the substance capable of inhibiting the activity of CSF-1 and/or nucleic acid, comprising at least one sequence coding for a substance capable of inhibiting the activity of CSF-1, is administered in advance such that the concentration of CSF-1 in the plasma of the host organism is less than 350 pg/ml at the time that the substance with at least one cytotoxic activity is administered.

The quantity of the combination product to be administered varies as a function of the different parameters, for example such as the nature of the substance capable of inhibiting the activity of CSF-1, the nature of the substance with at least one cytotoxic activity, the individual or disease to be treated, or the administration methods, or the host organ/tissue. For example, there are substance doses with at least one cytotoxic activity of 75 mg/kg/d of CDDP in one time and 500 mg/kg/d for 5 days in the case of cancer of the uterine cervix.

According to the invention, there are many possible administration methods. For example, there are the systemic, intragastric, sub-cutaneous, intracardiac, intramuscular, intravenous, intralymphatic, intraperitoneal, intratumoural, intranasal, intrapulmonary or intratracheal methods. For these last three embodiments, administration by aerosol or instillation is advantageous. Preferably, the oligonucleotide or the combination product according to the invention is administered by intralymphatic, intratumoural or peritumoural methods, in other words in an accessible tumour, at its periphery or in a lymphatic vessel or ganglion connected to the affected organ or the tumour.

The invention also relates to a method for the treatment of diseases by genic therapy, characterised in that a combination product according to the invention including at least one nucleic acid and/or at least one oligonucleotide is administered to host organism or cell needing such treatment.

Advantageously, when treating diseases by genic therapy, nucleic acid will include the following in its form to be administered, depending on the nature of the vector(s) used:
when the vector is of plasmidic origin, from 0.01 to 100 mg of DNA, preferably between 0.05 and 10 mg, and ideally between 0.5 and 5 mg;
when the vector is of viral origin, between 10⁴ and 10¹⁴ pfu (plaque forming units), advantageously between 10⁵ and 10¹³ pfu and preferably between 10⁵ and 10¹² pfu.

When the said treatment uses a substance with a UPRTase activity, it may be advantageous to administrate a second substance with a CDase activity. In this case, the administration of the UPRTase and CDase sequences may be simultaneous or consecutive, the order of administration being unimportant. According to such an embodiment, the treatment will also include an additional step by which pharmaceutically acceptable quantities of predrug will be administered to the host organism or cell, advantageously an analogue of cytosine and in particular of 5-FC. For illustration, a dose of 50 to 500 mg/kg/day may be used, with a preference for 200 mg/kg/day. This predrug may be administered according to standard practice and in advance, concomitant with or after the combination product according to this invention. The oral method is preferred. A single dose of predrug or repeated doses may be administered for a sufficiently long time to enable the production of toxic metabolite within the host organism or cell.

Possible applications include breast cancer, cancers of the ovary, the uterus (particularly cancers induced by papilloma viruses), cancers of the prostate, lung, bladder, liver, colon, pancreas, stomach, oesophagus, larynx, the central nervous system, and the blood (lymphomas, leukaemia, etc.), and bones (sarcomas).

According to one advantageous embodiment of the invention, the use or the treatment method according to the invention is associated with a prior or subsequent treatment of the patient by surgery (particularly by partial or complete ablation of the tumour) or by radiotherapy. In this particular case, the treatment according to the invention is applied before, concomitantly or after the said second treatment. Preferably, this treatment will be carried out after the second treatment.

Similarly, the efficiency of the intra-cell transfer of the oligonucleotide or the nucleic acid may advantageously be facilitated, for example by combination with an electroporation treatment (Vicat et al., 2000, Human Gene Therapy, 11, 909-916) and/or a treatment designed to modify the permeability of blood vessels in which the administration (WO 98/58542) is made or by any other methods described in the literature.

The purpose of the following examples is to illustrate the different purposes of this invention, and consequently they are not in any way limitative.

### EXAMPLE 1

Oligonucleotides:

All oligonucleotides used in this study (SEQ ID No. 2 to SEQ ID No. 5) were synthesised by Eurogentec (Seraing, Belgium) and purified by chromatography. There are two orders of modifications of oligonucleotides used in this study. Firstly, a modification of the phosphodiester skeleton to phosphorothioate. Secondly, a modification of the chemistry of bases, uridine (U) being replaced by a 5-(1-propynyl)-2′deoxyuridine (pdU) and cytosine C being replaced by a 5-1-propynyl)-2′-deoxycytidine (pdC). The substitution ratio of bases was limited to 50% of the contents of C and U.

The capacity of oligonucleotides used to hybridise themselves to SEQ ID No. 1 was checked in vitro by thermal denaturation test experiments. The formation of the oligonucleotide/SEQ ID No. 1 duplex was monitored by measuring absorbance of a solution containing 1 μm of oligonucleotide and 1 μm of SEQ ID No. 1 at 260 nm, as a function of the temperature. All experiments were done in a 10 mM buffer of cacodylate, 50 mM NaCl, pH 7.0.

Cell culture and Transfection of Oligonucleotides

Line NS2T2A1 is a line of cells from the human breast (Ma et al., Int. J. Cancer, 1998, 78: 112-119) obtained by mammoplasty, transformed by SV40. This line secretes CSF-1 linearly as a function of the cell growth.

The cells were cultivated in medium M5 (Gibco, France) complemented with 5% of -horse serum as described by Ma et al. (Int. J. Cancer, 1998, 78:112-119). Seeding was done in 6 well plates at a concentration, of 5×10⁵ cells/ml/well. Transfection by oligonucleotides was done 24 h later, when the cells were in the exponential growth phase and they had reached 60% to 80% of confluence.

Transfection by oligonucleotides was done using cytofectin GS-3815 (Glen research, Sterling. Va.). Oligonucleotides and cytofectin were diluted separately in polystyrene tubes with the opti-MEM medium (Gibco, France), and the cytofectin was mixed with an equal volume of diluted oligonucleotides. The mix was left for 15 minutes at ambient temperature to enable the formation of oligonucleotide/cytofectin complexes. Finally, the M5 medium was added to the solution to obtain final concentrations equal to 2.4 μg/ml of cytofectin and 250 nM of oligonucleotides. All transfections were done in the M5 medium and in a final volume of 1 ml. Oligonucleotide/cytofectin complexes were left in contact with cells for 24 h. All steps described above were done under sterile conditions.

All experiments were done in triplicate for each concentration of oligonucleotides.

Quantification of CSF-1

The quantity of CSF-1 secreted in the culture medium of NS2T2A1 cells was-measured by an ELISA test, 24 h after the beginning of the transfection, referring strictly to the protocol described by the supplier (R&D systems, Europe Ltd, UK). The results are expressed as a percentage of CSF-1 expressed by the transfected cells with respect to the CSF-1 expressed by a non-transfected cell line.

Results

The results obtained are grouped in the following table.

| | Hybridisation | |
|---|---|---|
| Oligonucleotide | region on SEQ ID | Percentage of CSF-1 |
| sequence | No. 1 | expressed |
| SEQ ID No. 2 | 135-152 | 70% |
| SEQ ID No. 3 | 284-301 | 39% |
| SEQ ID No. 4 | 341-358 | 50% |
| SEQ ID No. 5 | 409-426 | 90% |

These results clearly show that cells transfected by oligonucleotides according to the invention express less CSF-1 than cells not transfected by the said oligonucleotides.

1-33. (canceled)

34. A combination product comprising:
(i) at least one substance capable of inhibiting CSF-1 activity and/or at least one nucleic acid, comprising at least one sequence coding for a substance capable of inhibiting CSF-1 activity, and,
(ii) at least one substance having at least a cytotoxic activity and/or at least one nucleic acid, comprising at least one sequence coding for a substance having at least a cytotoxic activity,
characterised in that said oligonucleotide is selected from the group of oligonucleotides capable of hybridizing at the region between the nucleotide in position 121 and the nucleotide in position 450 inclusive of SEQ ID NO:1, between the nucleotide in position 131 and the nucleotide in position 391 inclusive of SEQ ID NO:1, between the nucleotide in position 135 and the nucleotide in position 152 inclusive of SEQ ID NO:1, between the nucleotide in position 284 and the nucleotide in position 301 inclusive of SEQ ID NO:1 or between the nucleotide in position 341 and the nucleotide in position 358 inclusive of SEQ ID NO:1.

35. The combination product according to claim 34, characterised in that said oligonucleotide comprises 8 to 30 nucleotides.

36. The combination product according to claim 35, characterised in that said oligonucleotide comprises 12 to 25 nucleotides.

37. The combination product according to claim 34, characterised in that the substance having at least a cytotoxic activity is selected from the substances interacting with DNA, antimetabolites, topoisomerase inhibitors, spindle agents and cytostatic agents.

38. The combination product according to claim 34, characterised in that the substance having at least a cytotoxic activity is a tumour-related antigen.

39. The combination product according to claim 34, characterised in that the substance having at least a cytotoxic activity is selected from cytokines, polypeptides showing a chemo-attraction activity, proteins coded by a suicide gene, anti-angiogenic protein factors and polypeptides showing a cellular apoptosis activation activity.

40. A formulation comprising at least one combination product, according to claim 34 and a pharmaceutically acceptable vehicle.

41. A method for the treatment of cancer comprising administering a therapeutically effective amount of a combination product according to claim 34 to a subject in need thereof.


SEQUENCE LISTING (5 sequences)

SEQ ID NO: 1 - DNA, 3939 bp, Homo sapiens
cctgggtcctctcggcgccagagccgctctccgcatcccaggacagcggtgcggccctcggccggggcgcccactccgcagcagccagcgagccagctgccccgtatgaccgcgccgggcgccgccgggcgctgccctcccacgacatggctgggctccctgctgttgttggtctgtctcctggcgagcaggagtatcaccgaggaggtgtcggagtactgtagccacatgattgggagtggacacctgcagtctctgcagcggctgattgacagtcagatggagacctcgtgccaaattacatttgagtttgtagaccaggaacagttgaaagatccagtgtgctaccttaagaaggcatttctcctggtacaagacataatggaggacaccatgcgcttcagagataacaccgccaatcccatcgccattgtgcagctgcaggaactctctttgaggctgaagagctgcttcaccaaggattatgaagagcatgacaaggcctgcgtccgaactttctatgagacacctctccagttgctggagaaggtcaagaatgtctttaatgaaacaaagaatctccttgacaaggactggaatattttcagcaagaactgcaacaacagctttgctgaatgctccagccaagatgtggtgaccaagcctgattgcaactgcctgtaccccaaagccatccctagcagtgacccggcctctgtctcccctcatcagcccctcgccccctccatggcccctgtggctggcttgacctgggaggactctgagggaactgagggcagctccctcttgcctggtgagcagcccctgcacacagtggatccaggcagtgccaagcagcggccacccaggagcacctgccagagctttgagccgccagagaccccagttgtcaaggacagcaccatcggtggctcaccacagcctcgcccctctgtcggggccttcaaccccgggatggaggatattcttgactctgcaatgggcactaattgggtcccagaagaagcctctggagaggccagtgagattcccgtaccccaagggacagagctttccccctccaggccaggagggggcagcatgcagacagagcccgccagacccagcaacttcctctcagcatcttctccactccctgcatcagcaaagggccaacagccggcagatgtaactgctacagccttgcccagggtgggccccgtgatgcccactggccaggactggaatcacaccccccagaagacagaccatccatctgccctgctcagagaccccccggagccaggctctcccaggatctcatcactgcgcccccaggccctcagcaacccctccaccctctctgctcagccacagctttccagaagccactcctcgggcagcgtgctgccccttggggagctggagggcaggaggagcaccagggatcggacgagccccgcagagccagaagcagcaccagcaagtgaaggggcagccaggcccctgccccgttttaactccgttcctttgactgacacaggccatgagaggcagtccgagggatcctccagcccgcagctccaggagtctgtcttccacctgctggtgcccagtgtcatcctggtcttgctggctgtcggaggcctcttgttctacaggtggaggcggcggagccatcaagagcctcagagagcggattctcccttggagcaaccagagggcagccccctgactcaggatgacagacaggtggaactgccagtgtagagggaattctaagctggacgcacagaacagtctcttcgtgggaggagacattatggggcgtccaccaccacccctccctggccatcctcctggaatgtggtctgccctccaccagagctcctgcctgccaggactggaccagagcagccaggctggggcccctctgtctcaacccgcagacccttgactgaatgagagaggccagaggatgctccccatgctgccactatttattgtgagccctggaggctcccatgtgcttgaggaaggctggtgagcccggctcaggaccctcttccctcaggggctgcagcctcctctcactcccttccatgccggaacccaggccagggacccaccggcctgtggtttgtgggaaagcagggtgcacgctgaggagtgaaacaaccctgcacccagagggcctgcctggtgccaaggtatcccagcctggacaggcatggacctgtctccagacagaggagcctgaagttcgtggggcgggacagcctcggcctgatttcccgtaaaggtgtgcagcctgagagacgggaagaggaggcctctgcacctgctggtctgcactgacagcctgaagggtctacaccctcggctcacctaagtccctgtgctggttgccaggcccagaggggaggccagccctgccctcaggacctgcctgacctgccagtgatgccaagagggggatcaagcactggcctctgcccctcctccttccagcacctgccagagcttctccagcaggccaagcagaggctcccctcatgaaggaagccattgcactgtgaacactgtacctgcctgctgaacagcctccccccgtccatccatgagccagcatccgtccgtcctccactctccagcctctccccagcctcctgcactgagctggcctcaccagtcgactgagggagcccctcagccctgaccttctcctgacctggcctttgactccccggagtggagtggggtgggagaacctcctgggccgccagccagagccgctctttaggctgtgttcttcgcccaggtttctgcatcttccactttgacattcccaagagggaagggactagtgggagagagcaagggaggggagggcacagacagagagcctacagggcgagctctgactgaagatgggcctttgaaatataggtatgcacctgaggttgggggagggtctgcactcccaaaccccagcgcagtgtcctttccctgctgccgacaggaacctggggctgagcaggttatccctgtcaggagccctggactgggctgcatctcagccccacctgcatggtatccagctcccatccacttctcacccttctttcctcctgaccttggtcagcagtgatgacctccaactctcacccaccccctctaccatcacctctaaccaggcaagccagggtgggagagcaatcaggagagccaggcctcagcttccaatgcctggagggcctccactttgtggccagcctgtggtgctggctctgaggcctaggcaacgagcgacagggctgccagttgcccctgggttcctttgtgctgctgtgtgcctcctctcctgccgccctttgtcctccgctaagagaccctgccctacctggccgctgggccccgtgactttcccttcctgcccaggaaagtgagggtcggctggccccaccttccctgtcctgatgccgacagcttagggaagggcactgaacttgcatatggggcttagccttctagtcacagcctctatatttgatgctagaaaacacatatttttaaatggaagaaaaataaaaaggcattcccccttcatccccctaccttaaacatataatattttaaaggtcaaaaaagcaatccaacccactgcagaagctctttttgagcacttggtggcatcagagcaggaggagccccagagccacctctggtgtcccccaggctacctgctcaggaaccccttctgttctctgagaactcaacagaggacattggctcacgcactgtgagattttgtttttatacttgcaactggtgaattattttttataaagtcatttaaatatctatttaaaagataggaagctgcttatatatttaataataaaagaagtgcacaagctgccgttgacgtagctcgag

SEQ ID NO: 2 - RNA, 18 nt, Artificial sequence
  Description of artificial sequence synthetic oligonucleotide
gggagggugcuguaccga

SEQ ID NO: 3 - RNA, 16 nt, Artificial sequence
  Description of artificial sequence synthetic oligonucleotide
ucuggagcagguuaau

SEQ ID NO: 4 - RNA, 18 nt, Artificial sequence
  Description of artificial sequence synthetic oligonucleotide
acacgauggaauucuucc

SEQ ID NO: 5 - RNA, 18 nt, Artificial sequence
  Description of artificial sequence synthetic oligonucleotide
uuguggcgguuaggguag